(19) United States
(12) Patent Application Publication
(10) Pub. No.: US 2025/0186702 A1
(43) Pub. Date: Jun. 12, 2025

(54) SAMPLE COLLECTION CUP AND SYRINGE

(71) Applicant: Mosie, Inc., Austin, TX (US)

(72) Inventors: Maureen Brown, Austin, TX (US); Marc Brown, Austin, TX (US)

(21) Appl. No.: 18/972,212

(22) Filed: Dec. 6, 2024

Related U.S. Application Data
(60) Provisional application No. 63/607,022, filed on Dec. 6, 2023.

(51) Int. Cl. A61M 5/31; A61B 10/00; A61B 17/43

(52) U.S. Cl. CPC A61M 5/3134; A61B 10/0058; A61B 17/43

(57) ABSTRACT

A device may include a top region defining an opening having a top diameter. A device may include a bottom region opposite the top region and defining a base. A device may include an outer wall extending from the top region to the bottom region. A device may include an inner wall coupled to the outer wall and extending inward away from the outer wall and downward toward the base, the inner wall defining an accumulation area and having a profile configured to direct fluid into the accumulation area.

### CROSS-REFERENCE TO RELATED APPLICATION(S)

This application claims priority to priority to U.S. Provisional Patent Application No. 63/607,022 filed on Dec. 6, 2023, the entire contents of which are incorporated herein by reference.

### BACKGROUND

The present invention relates to a fluid collection cup and, more particularly, to a fluid collection cup having an inner surface and an outer surface.

Infertility is a common problem among couples. An evaluation by a doctor is often recommended after a period of time (such as one year) of unprotected intercourse without achieving conception. While it is sometimes possible to identify correctable issues that may be preventing conception, some couples pursue expensive medical solutions, such as in vitro fertilization at a fertility clinic. A personal insemination syringe for home use provides an effective and affordable alternative to expensive and complicated medical procedures.

Sample collection cups are well known in the medical field as a way to collect, hold, and transport a bodily fluid such as semen, blood, or urine. The fluid may be extracted from the collection cup by a syringe for testing or other medical purposes. For example, semen may be extracted from the collection cup to be used for artificial insemination. In some uses (e.g., in artificial insemination) it is beneficial to extract all the fluid from the collection cup. Existing collection cups typically fall short of allowing full extraction of the fluid from the cup.

### SUMMARY

In some aspects, the techniques described herein relate to a sample collection cup including a top region defining an opening having a top diameter, a bottom region opposite the top region and defining a base, an outer wall extending from the top region to the bottom region, and an inner wall coupled to the outer wall and extending inward away from the outer wall and downward toward the base. The inner wall defines an accumulation area and having a profile configured to direct fluid into the accumulation area.

In some aspects, the techniques described herein relate to a sample collection cup including a top region defining an opening and a top diameter, a bottom region opposite the top region, a transition connecting the top region and the bottom region, and at least one wall including an outer surface extending from the top region to the bottom region and an inner surface extending from the transition toward the bottom region. The inner surface has a lowermost flat section.

In some aspects, the techniques described herein relate to a sample collection system including a syringe including a body elongated along a longitudinal axis, the body having an open end and an opening defined on a distal end of the body opposite the open end, a plunger configured to extend into the open end and along the longitudinal axis toward the opening, and a cup including a first wall defining an outer profile and a second wall inward of the first wall and defining an inner profile different from the outer profile. The inner profile is shaped to accumulate fluid in the cup. The plunger is configured to draw fluid accumulated in the cup into the syringe.

Other aspects of the invention will become apparent by consideration of the detailed description and accompanying drawings.

### BRIEF DESCRIPTION OF THE DRAWINGS

FIG. 1 is a perspective view of an apparatus including a syringe with a nub, in accordance with certain embodiments of the present invention.

FIG. 2 is a perspective view of an apparatus including a syringe with a nub, in accordance with certain embodiments of the present invention.

FIG. 3 is a cross-sectional view of an apparatus including a syringe with a nub, in accordance with certain embodiments of the present invention.

FIG. 4 is a cross-sectional view of an apparatus including a syringe and a sheath including a nub, in accordance with certain embodiments of the present invention.

FIG. 5 is a cross-sectional view of a sheath including a nub and a threaded attachment, in accordance with certain embodiments of the present invention.

FIG. 6 is a cross-sectional view of a sheath including a nub and a threaded attachment, in accordance with certain embodiments of the present invention.

FIG. 7 is a front view of a distal end of the sheath of FIG. 6, in accordance with certain embodiments of the present invention.

FIG. 8 is a front view of a distal end of a sheath, similar to the sheath of FIG. 6, in accordance with certain embodiments of the present invention.

FIG. 9 is a cross-sectional view of a sheath including a nub, in accordance with certain embodiments of the present invention.

FIG. 10 is a cross-sectional view of a sheath including a nub, m accordance with certain embodiments of the present invention.

FIG. 11 is a side view of an apparatus including a syringe and a cap including a nub, in accordance with certain embodiments of the present invention.

FIG. 12 is a cross-sectional view of the cap of FIG. 11 including an attachment mechanism, in accordance with certain embodiments of the present invention.

FIG. 13 is a cross-sectional view of the cap of FIG. 11 including an attachment mechanism, in accordance with certain embodiments of the present invention.

FIG. 14 is a side view of a cap configured to couple to a syringe, in accordance with certain embodiments of the present invention.

FIG. 15 is a flow diagram of a method of forming a sheath configured to receive a syringe, in accordance with certain embodiments of the present invention.

FIG. 16A is a side-view of a syringe including multiple openings, in accordance with certain embodiments of the present invention.

FIG. 16B is a cross-sectional view of the syringe of FIG. 16A taken along line A-A, in accordance with certain embodiments of the present invention.

FIG. 17 is an exploded view and a perspective view of the syringe of FIG. 16A, in accordance with certain embodiments of the present invention.

FIG. 18A is a front view of the syringe of FIG. 16A, in accordance with certain embodiments of the present invention.

FIG. 18B is a perspective view of the syringe of FIG. 16A, in accordance with certain embodiments of the present invention.

FIG. 19A is a side view of a syringe including a nub, in accordance with certain embodiments of the present invention.

FIG. 19B is cross-sectional view of the syringe of FIG. 19A taken along line B-B, in accordance with certain embodiments of the present invention.

FIG. 20A is a front view of the syringe of FIG. 19 A, in accordance with certain embodiments of the present invention.

FIG. 20B is a perspective view of the syringe of FIG. 19A, in accordance with certain embodiments of the present invention.

FIGS. 21A-21D are front views of the syringe of FIG. 19A, in accordance with certain embodiments of the present invention.

FIGS. 22A-22D are front views of the syringe of FIG. 19A, in accordance with certain embodiments of the present invention.

FIG. 23 is a side cross-sectional view of the syringe with sectional lines and a front view of the syringe with sectional lines.

FIG. 24 is a perspective view of a collection cup embodying the present invention.

FIG. 25 is a perspective view of the collection cup of FIG. 24 with the lid removed.

FIG. 26 is a side view of the collection cup of FIG. 24.

FIG. 27 is a top view of the collection cup of FIG. 24.

FIG. 28 is an exploded view of the collection cup of FIG. 24.

FIG. 29 is a side view of the collection cup of FIG. 24 with the lid removed.

FIGS. 30A-30B is a section view of the collection cup of FIG. 29 taken along A-A.

FIGS. 31A-31B is a detailed view of the region B in FIGS. 30A-30B.

FIG. 32 is a front view of another exemplary collection cup embodying the present invention.

FIGS. 33A-33B are a section view of the collection cup of FIG. 32.

### DETAILED DESCRIPTION

Before any embodiments of the disclosed technology are explained in detail, it is to be understood that the technology is not limited in its application to the details of construction and the arrangement of components set forth in the following description or illustrated in the following drawings. The technology is capable of other embodiments and of being practiced or of being carried out in various ways. Also, it is to be understood that the phraseology and terminology used herein is for the purpose of description and should not be regarded as limiting. Any implementation described herein as “exemplary” is not necessarily to be construed as preferred or advantageous over other implementations. The detailed description uses numerical and letter designations to refer to features in the drawings. Like or similar designations in the drawings and description have been used to refer to like or similar parts of the embodiments of the technology.

As used herein, the terms “first”, “second”, and “third” may be used interchangeably to distinguish one component from another and are not intended to signify location or importance of the individual components, unless otherwise context dictates otherwise. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises.” “comprising,” “includes,” “including.” “has,” “having” or any other variation thereof, are intended to cover a non-exclusive inclusion. For example, a process, method. article, or apparatus that comprises a list of features is not necessarily limited only to those features but may include other features not expressly listed or inherent to such process. method, article. or apparatus. Further. unless expressly stated to the contrary, “or” refers to an inclusive-or and not to an exclusive-or. For example. a condition A or B is satisfied by any one of the following: A is true (or present) and B is false (or not present), A is false (or not present) and B is true (or present), and both A and B are true (or present).

Terms of approximation, such as “about,” “generally,” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction (e.g., clockwise, or counterclockwise).

Benefits, other advantages, and solutions to problems may be described below with regard to specific embodiments. However, the benefits, advantages, solutions to problems, and any feature(s) that may cause any benefit, advantage, or solution to occur or become more pronounced are not to be construed as a critical, required, or essential feature of any or all the claims.

In the following discussion, the same reference numbers are used in the various embodiments to indicate the same or similar elements. Embodiments of an apparatus are disclosed below that include a substantially cylindrical body portion and a nub that extends from a distal end of the body portion. The cylindrical body portion may be a syringe, a sheath sized to receive a syringe, or cap configured to couple to a syringe. The nub may have a rounded shape, such as a hemispherical shape, a ring shape (with rounded edges), an elliptical shape (with rounded edges), a bulbous shape, or some other rounded shape. In some embodiments, the nub may have a diameter that is less than a cross-sectional diameter of the syringe. Further, the edges of the syringe, the sheath, the cap, or any combination thereof, may be rounded, presenting a smooth transition from the elongate body portion to the distal end. In some embodiments, portions of the syringe, portions of the cap, portions of the sheath, or any combination thereof may be formed from a first material, while other portions of the syringe, the sheath, the cap, the nub, or any combination thereof may be formed from a second material. In some embodiments, the second material may be more flexible or malleable than the first material.

In some embodiments, the apparatus may be used for artificial insemination by filling a fluid canal of a barrel of the syringe with seminal fluid, and by inserting the apparatus into the vaginal canal to deliver seminal fluid through the vaginal canal to the cervix of the woman The nub may provide tactile feedback to the woman by allowing her to feel when the distal end of the apparatus is touching the cervix. Further, the feedback provided by the nub may allow the woman to adjust the position of the apparatus relative to the cervix prior to depression of the plunger of the syringe to deliver the seminal fluid.

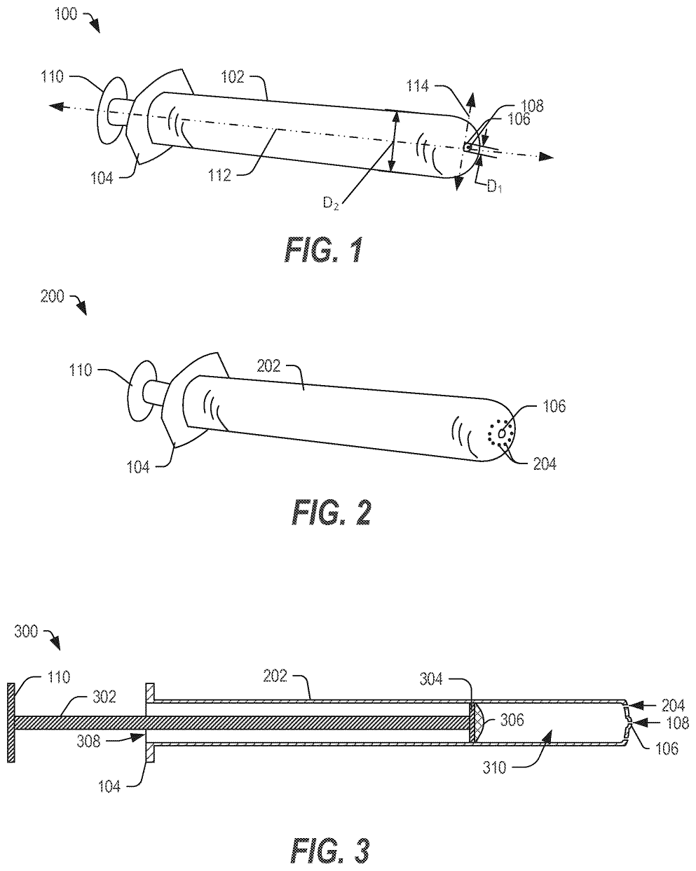

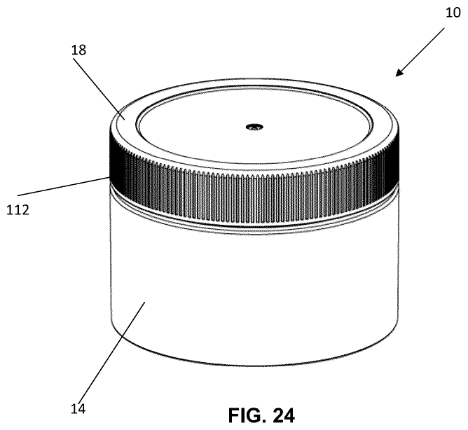

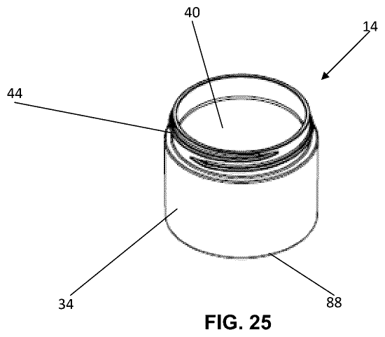

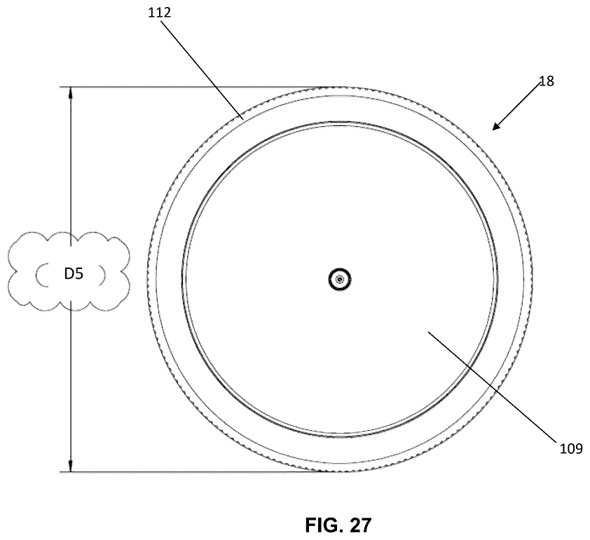

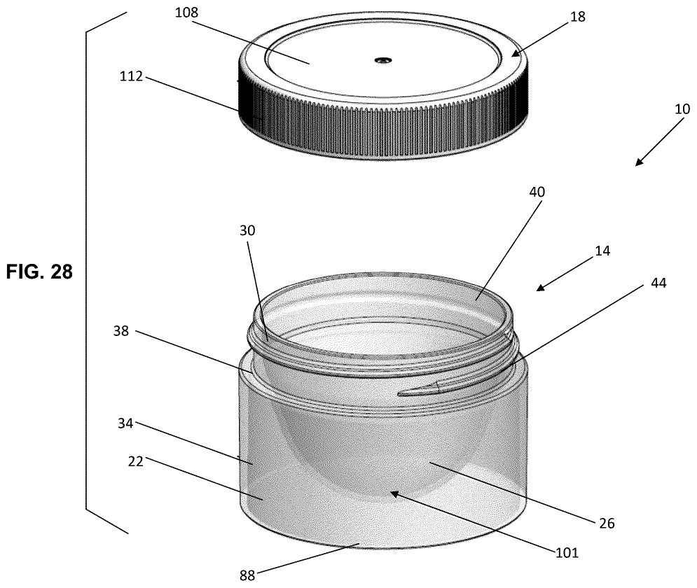

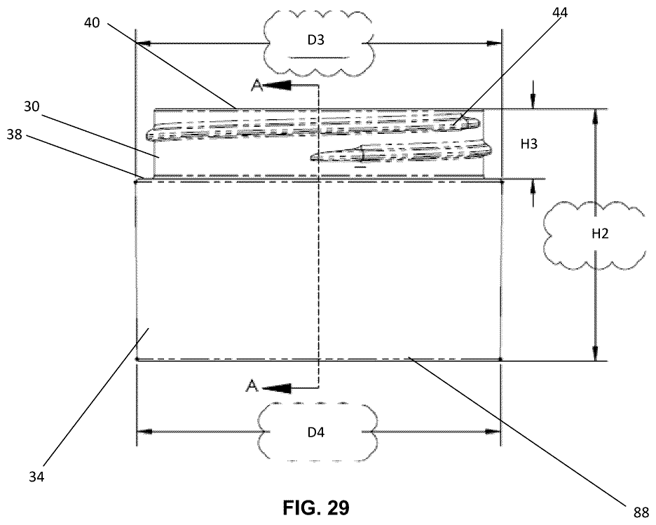

FIGS. 1-23 illustrate embodiments of exemplary syringes that may be used with a collection cup (e.g., a sample collection cup illustrated in FIGS. 24-33B) that has an interior shape to facilitate easy collection of fluid and easy withdrawal via a syringe. Exemplary syringes contemplated herein include the syringes described and illustrated in U.S. application Ser. No. 17/834,481 (assigned to Mosie Inc.), filed on Jun. 7, 2022, published as US20230056164, and titled “APPARATUS INCLUDING A CYLINDRICAL BODY AND NUB”, the entire contents of which are incorporated by reference herein. The syringe may have a substantially cylindrical profile (e.g., a tubular profile) along its length. In one non-limiting example, the profile may have a small or slight taper toward the tip or distal end of the syringe. For example, the syringe may be substantially cylindrical with a first diameter adjacent the flanges and a second diameter adjacent the distal end that is smaller than the first diameter. The smooth transition between the first diameter and the second diameter may be located anywhere along the length of the tubular profile (e.g., adjacent a middle of the tubular profile along the longitudinal axis between the flanges and the distal end).

FIG. 1 is a perspective view of an apparatus 100 including a syringe 102 with a nub 106 according to some embodiments. The syringe 102 may include a body having a proximal end with a flange 104 and may define a cavity sized to receive a plunger 110 to draw fluid into the cavity and to propel fluid through the opening 108. The flange 104 may be used as leverage by a user when depressing the plunger 110. Further, the body of the syringe 102 may include a distal end with the nub 106. The distal end of the body of the syringe 102 may also include an opening 108 to allow fluid flow. In the illustrated example, the opening 108 extends through a corresponding opening in the nub 106; however, the nub 106 and the opening 108 may be offset from one another. In some embodiments, the opening 108 may be offset from a center of the distal end of the syringe 102. In some embodiments, the nub 106 may be offset from the center of the distal end. In some embodiments, the nub 106 and the opening 108 may be offset from the center of the distal end.

In some embodiments, the body of the syringe 102 may have a substantially cylindrical shape (or tubular shape) forming a fluid conduit and having rounded edges at the distal end. Further, the nub 106 may have a substantially semi-spherical shape and may extend outward from the distal end of the syringe 102 proximate to a longitudinal axis 112 of the syringe 102. In some embodiments, the nub 106 may have a diameter (D1) that is orthogonal to the longitudinal axis 112 of the syringe 102 and that is smaller than a diameter (D2) of the body of the syringe 102. In some embodiments, the body of the syringe 102 may be formed from a first material, and the nub 106 may be formed from a second material. In some embodiments, the body of the syringe 102 and the nub 106 may be formed from a unitary piece of material (such as molded antibacterial plastic).

In some embodiments, a user may draw fluid into a cavity within the body of the syringe 102 by pulling the plunger 110. The user may then insert the syringe 102 into the vaginal cavity and depress the plunger 110 to dispense the fluid through opening 108. The nub 106 may provide feed-back to the woman to allow her to feel the position of the distal end of the syringe 102 against her cervix prior to dispensing the fluid.

In some embodiments, the rounded edges of the distal end of the body of the syringe 102 and the rounded shape of the nub 106 provide a substantially smooth and relatively comfortable feel as compared to a standard syringe that has corners and a pointed (though needle-less) end. While a conventional syringe may have edges or corners that can be sharp or abrasive, the syringe 102 and the nub 106 are rounded to provide smooth edges.

While the illustrated example of FIG. 1 included a single opening that extends through the nub 106, it should be understood that the distal end of the syringe 102 may include one or more openings to allow fluid passage to and from the cavity within the body of the syringe 102 and the outside environment. One possible example of a syringe that may include multiple openings is described below with respect to FIG. 2.

FIG. 2 is a perspective view of an apparatus 200 including a syringe 202 with a nub 106 according to some embodiments. The syringe 202 may include the flange 104 and may include a cavity sized to receive the plunger 110. The syringe 202 further may include multiple openings 204 extending from the cavity to the outside environment. In the illustrated example, the openings 204 are distributed about the nub 106 and are offset from the nub 106. In some embodiments, additional openings 204 or fewer openings may be provided. In some embodiments, the openings 204 may be distributed circumferentially along the sidewalls of the body of the syringe 202 near the distal end. Further, in some embodiments, the openings 204 may align with corresponding openings that extend through the nub 106.

In the illustrated examples of FIGS. 1 and 2, the syringe 102 and 202 may be formed from a first material, such as a substantially rigid, anti-bacterial and anti-microbial plastic material, while the nub 106 may be formed from a second material, that may also have anti-bacterial and anti-microbial properties, but that may be more elastic or malleable than the first material. Additionally, in some embodiments, the nub 106 may be attached to or coupled to the syringe 102, 202. In some embodiments, the nub 106 may be integrally formed with the syringe 102, 202. An example of some embodiments of the syringe 202 having an integrally formed nub 106 and including the opening 108 is described below with respect to FIG. 3.

FIG. 3 is a cross-sectional view of an apparatus 300 including a syringe 202 with a nub 106 according to some embodiments. The syringe 202 may include a body portion that defines a cavity 308 that extends from a proximal end, which may include the flange 104, to a distal end, which may include openings 204, and the nub 106. Additionally, in some embodiments, the nub 106 also may include an opening 108. The openings 108 and 204 extend through the distal end of the body of the syringe 202 to permit fluid flow between a fluid area 310 and the environment.

The apparatus 300 further may include the plunger 110 including a rod portion 302 and an end portion 304. A gasket or seal 306 may be coupled to the end portion 304 to provide a fluid seal to prevent fluid flow from the fluid area 310 toward the body portion 302 and to draw fluid through the openings 108 and 204 into the fluid area 310 or to drive fluid from the fluid area 310 through the openings 108 and 204.

In some embodiments, the nub 106 may be part of the distal end of the body of the syringe 202. In some embodiments, the nub 106 may omit the opening 108 and the openings 204 may be included. In some embodiments, the openings 204 may be omitted and the opening 108 may be included.

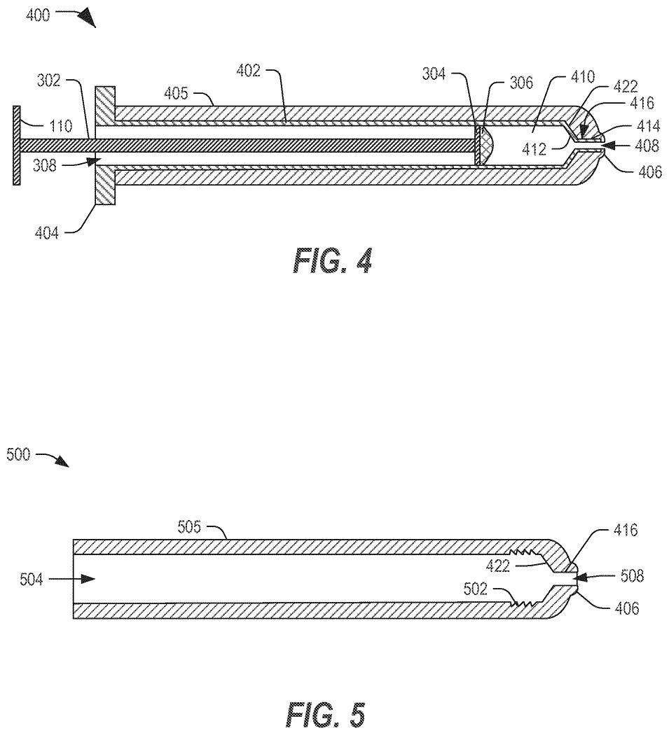

In some embodiments, an outer sheath or covering may be provided that may be adapted to cover a needle-less syringe. The outer sheath may be configured to receive the syringe and to provide a rounded distal end and a nub. An example of an apparatus including a sheath is described below with respect to FIGS. 4-10 according to some embodiments.

FIG. 4 is a cross-sectional view of an apparatus 400 including a syringe 402 and a sheath 405 including a nub 406 according to some embodiments. The syringe 402 may include a body portion defining the cavity 308 sized to receive a plunger 110 having a rod portion and an end portion 304 that is coupled to a gasket or seal 306. The syringe 402 may further include a flange 404, a hub portion 412, and a nose portion.

414. The nose portion 414 may define an opening for fluid passage from a fluid area 410 and the external environment. The sheath 405 may include a stop portion 422 configured to contact the hub portion 212 of the syringe 402 to seat the syringe 402 within the sheath 405. The sheath may be a substantially cylindrical (or tubular member) defining a cavity sized to receive the syringe 402. The sheath 405 may also include an opening 416 configured to receive the neck portion 414 of the syringe 402. The sheath 405 may further include a nub 406 that extends outward from a distal end of the sheath 405. The nub 406 may have a rounded, substantially hemispherical, ring, elliptical, or other rounded shape that may partially define the opening 416 that is configured to align with a corresponding opening 108 through the neck portion 414 to allow fluid passage.

In some embodiments, the sheath 405 may be formed from a first material, and the syringe 402 may be formed from a second material. The first material may be more elastic, more malleable, softer, or any combination thereof relative to the second material. In some embodiments, the sheath 405 may have a substantially cylindrical shape without edges and may define an opening on a proximal end that is sized to receive a syringe 402. The sheath 405 may include one or more openings configured to align with an opening on the syringe. Further, in some embodiments, the surface of the sheath 405 may have some texture or uneven areas. Additionally, in some embodiments, the sheath 405 may include an attachment element, such as threads to mate with corresponding threads on an outer surface of a syringe, a flange element configured to mate with a corresponding recess on the syringe, some other attachment structure, or any combination thereof. An example of a sheath that may include an attachment structure is described below with respect to FIG. 5.

FIG. 5 is a cross-sectional view 500 of a sheath 505 including a nub 406 and a threaded attachment 502 according to some embodiments. The sheath 505 may define a cavity 504 sized to receive a syringe and may include an opening 508 configured to align to a corresponding opening 408 of a syringe. The sheath 505 further may include an attachment feature 502. In the illustrated example, the attachment feature 502 may be threads configured to fit corresponding threads on an exterior surface of the syringe. The user may turn the syringe within the cavity 504 in order to secure the sheath 505 to the syringe.

In some embodiments, the size of the opening 416 may be sufficiently narrow to apply a hoop stress on the neck portion 414 of the syringe 402 to secure the sheath 505 to the syringe. In some embodiments, an extension on an inner surface of the sheath 505 may be configured to mate with a corresponding recess on an exterior surface of the syringe to secure the sheath 505 to the syringe.

In some embodiments, the sheath 505 may be formed from a medical grade silicone. In some embodiments, the sheath 505 may be formed from a medical device material, such as a polymer designed to support medical applications and to maintain its material properties even after sterilization. In some embodiments, the sheath 505 may be formed from a flexible, substantially malleable material that may be of a different material from that of the syringe.

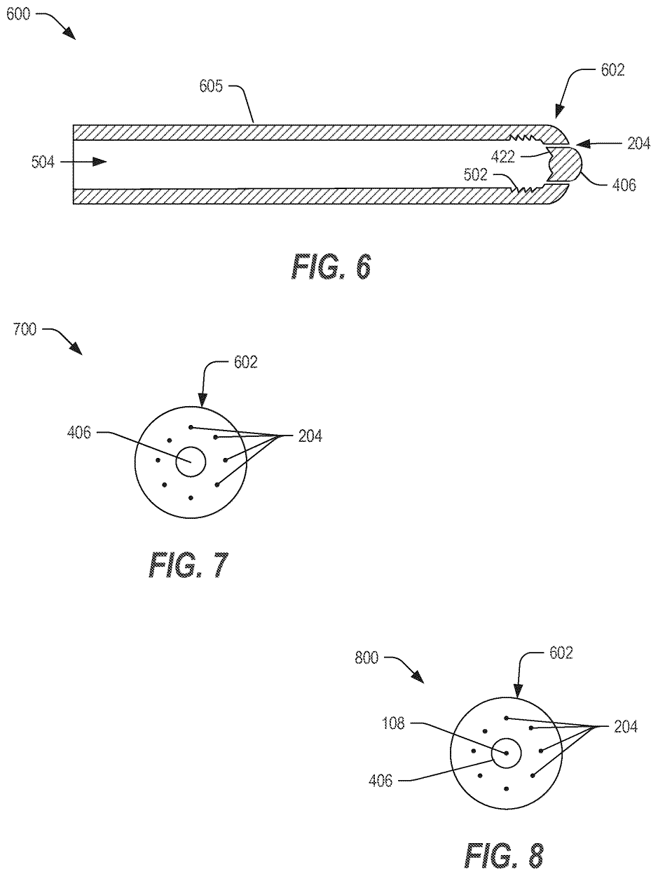

FIG. 6 is a cross-sectional view 600 of a sheath 605 including a nub 406 and a threaded attachment 502 according to some embodiments. The sheath 605 defines a cavity 504 sized to receive a syringe and may include a threaded attachment feature 502 configured to mate with a corresponding feature on an outside surface of the syringe to secure the syringe within the sheath 605. The sheath 605 may include a nub 406 that extends from a distal end 602 of the sheath 605. The sheath 605 may include openings 204 about a periphery of the nub 406.

FIG. 7 is a front view 700 of a distal end 602 of the sheath 605 of FIG. 6 according to some embodiments. The distal end 602 may include openings 204 and nub 406. While the openings 204 are distributed around the nub 406 of the distal end 602 approximately midway between the nub 406 and the peripheral edge of the distal end 602, in some embodiments, the openings 204 may be positioned closer to the peripheral edge or closer to the nub 406. Further, in some embodiments, additional openings or fewer openings may be provided. One possible example of a distal end that may include an additional opening is described below with respect to FIG. 8.

FIG. 8 is a front view 800 of a distal end 602 of a sheath, similar to the sheath 605 of FIG. 6, according to some embodiments. The front view 800 may include the opening 108 that extends through a center of the nub 406 in addition to the openings 204.

In some embodiments, the nub 406 may be positioned at approximately a center of the distal end 602. In some embodiments, the nub 406 may be offset from a center of the distal end 602. The opening 108 may be aligned to a longitudinal axis of a syringe.

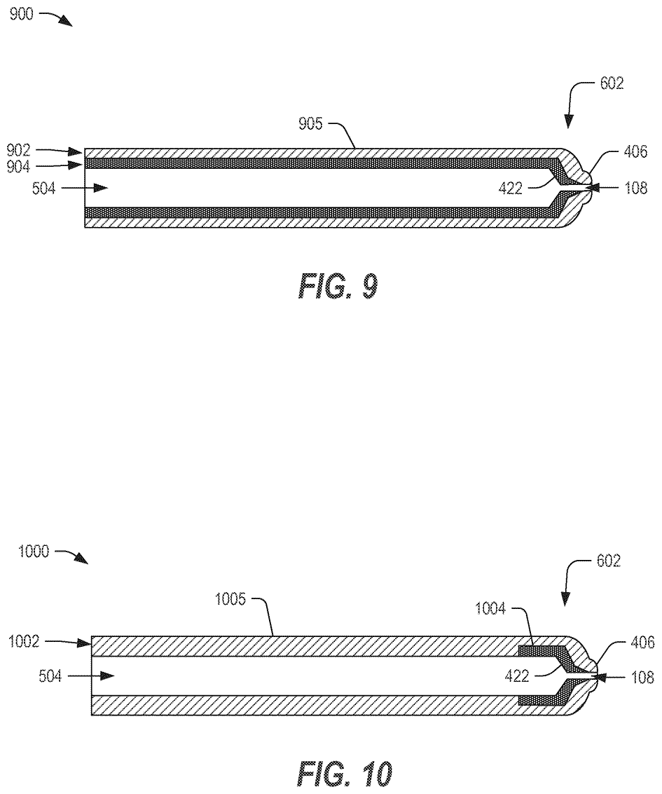

FIG. 9 is a cross-sectional view 900 of a sheath 905 including a nub 406 according to some embodiments. The sheath 905 may include multiple layers including an outer layer 902 and an inner layer 904. The sheath 905 may define a cavity or opening 504 sized to receive a syringe. The outer layer 902 may include the nub 406. In some embodiments, the inner layer 904 and the outer layer 902 may cooperate to form the nub 406. In some embodiments, the inner layer 904 may be formed from a first material and the outer layer 902 may be formed form a second material. In some embodiments, the first material may be more rigid than the second material. While the illustrated example of FIG. 9 depicts the inner layer extending from the opening of the proximal end along its entire length to the opening of the distal end 602, in some embodiments, the inner layer may extend only a portion of the length of the sheath 905. One possible example of a sheath having an inner layer that extends for only a portion of the length of the sheath is described below with respect to FIG. 10.

FIG. 10 is a cross-sectional view of a sheath 1005 including a nub 406 according to some embodiments. The sheath 1005 may include multiple layers including a first layer 1002 that extends about an entire periphery of the sheath 1005. The sheath 1005 may further include an inner layer 1004 that extends over a portion of an interior surface of the sheath 1005. In some embodiments, the inner layer 1004 may extend over the stop portion 422 of the sheath 1005, providing a relatively rigid seat against which the syringe may be positioned without over-stressing the distal end 602 of the sheath 1005.

In some embodiments, the nub 406 may include an opening that extends therethrough, and the nub 406 may be positioned at approximately a center of the distal end 602. In some embodiments, the nub 406 may be offset from a center axis of the sheath 1005. In some embodiments, the nub 406 may extend at least partially over the opening 108 to prevent the fluid from dispensing in a direct stream out of the opening 108, and dispersing the fluid over a larger spray area than a fluid stream would otherwise provide.

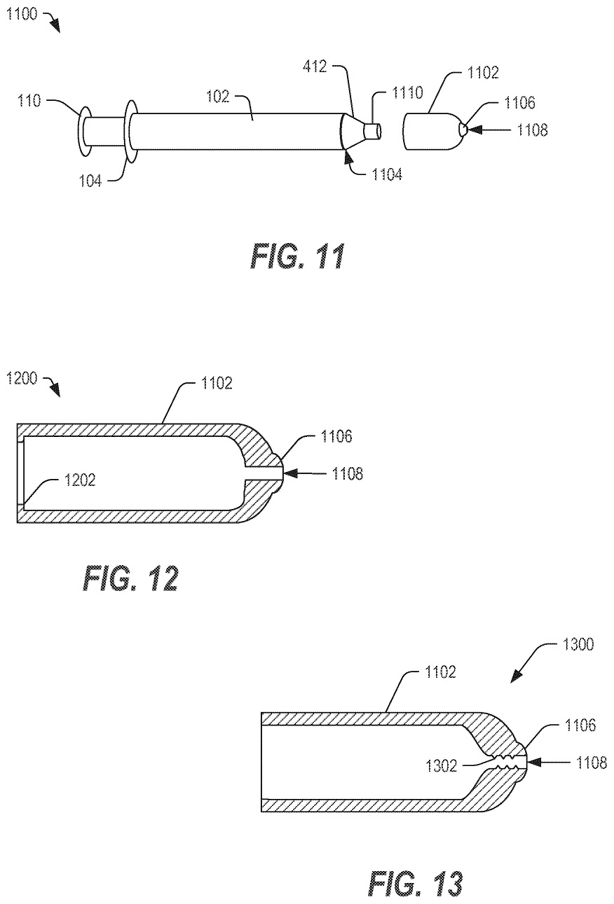

While the embodiments described above included a syringe with a nub and a sheath with a nub, in some embodiments, the nub may be provided on a cap configured to fit onto a distal end of a syringe. Examples of such embodiments are described below with respect to FIGS. 11-14.

FIG. 11 is a side view of an apparatus 1101 including a syringe 102 and a cap 1102 including a nub 1106 according to some embodiments. The syringe 102 may include the plunger 110 and the flange 104. The syringe 102 may further include a hub portion 412 and a neck portion 1110 that extends from the hub portion 412 and through which the opening 108 may extend. The syringe 102 may further include an attachment feature 1105 configured to mate with a corresponding attachment feature of the cap 1102.

In some embodiments, the cap 1102 may be a substantially cylindrical shape (or tubular shape) having a rounded end that may include a nub 1106 and an opening 1108 that extends through the cap 1102. The cap 1102 may include an attachment feature on an interior surface of the cap 1102 that may be configured to couple to the attachment feature 1105 on the syringe 102 to secure the cap 1102 to the syringe 102. In some embodiments, the cap 1102 may include an opening 1108 configured to align to the opening 108 of the neck 1110 of the syringe 102.

FIG. 12 is a cross-sectional view 1200 of the cap 1102 of FIG. 11 including an attachment mechanism 1202 according to some embodiments. The attachment mechanism 1202 may be configured to mate with a corresponding recess 1104 of the syringe 102 in a manner that may be similar to a pen cap coupling to a pen. In some embodiments, the attachment feature 1202 may be a flange or other structure that extends from an inner surface of the cap 1102. In some embodiments, the attachment feature 1202 may extend about an entire inner circumference of the cap 1102. In some embodiments, the attachment feature 1202 may extend about a portion of the inner circumference of the cap 1102.

FIG. 13 is a cross-sectional view 1300 of the cap 1102 of FIG. 11 including an attachment mechanism 1302 according to some embodiments. In some embodiments, the attachment mechanism 1302 may be located at a stop portion of the cap 1102. In some embodiments, the attachment mechanism 1302 may be located within an opening 1108 of the cap 1102. The attachment mechanism 1302 may be a threaded portion configured to mate with a corresponding threaded portion of a syringe to secure the cap 1102 to the syringe.

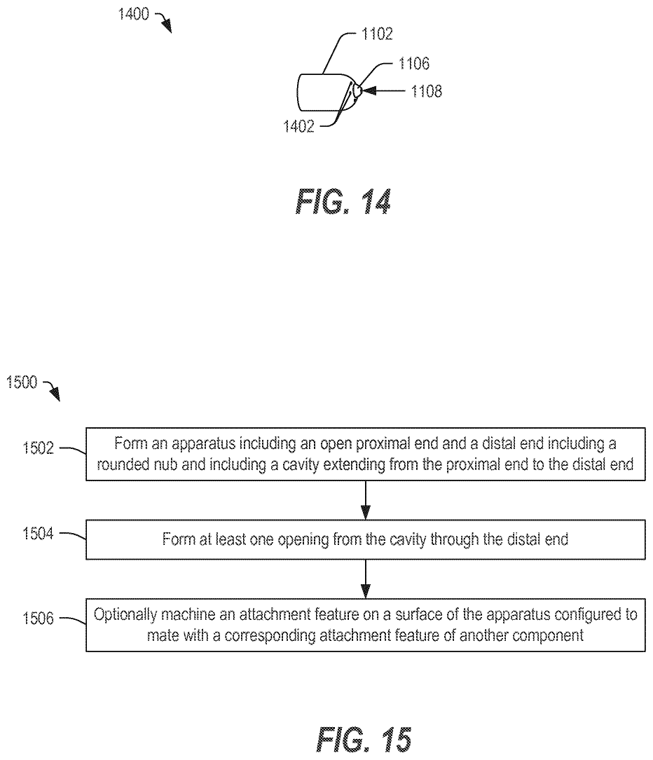

While the cap 1102 depicted in FIGS. 11-13 may include the opening 1108 located at a center of the rounded end of the cap 1102, it should be appreciated that, in some embodiments, the opening 1108 may be offset from a center axis of the cap 1102. In some embodiments, the cap 1102 may include multiple openings as described below with respect to FIG. 14.

FIG. 14 is a side view 1400 of a cap 1102 configured to couple to a syringe according to some embodiments. The cap 1102 may include a nub 1106 and an opening 1108 that extends through the nub 1106. Additionally, the cap 1102 may include multiple openings 1402, which may be arranged circumferentially about the nub 1106. Within the cap 1102, the openings 1402 may be coupled to an opening 108 of the syringe by conduits extending from the opening 108 to the openings 1402.

FIG. 15 is a flow diagram of a method 1500 of forming a sheath configured to receive a syringe according to some embodiments. At 1502, an apparatus may be formed that may include an open proximal end and a distal end including a rounded nub and that may include a cavity extending from the proximal end to the distal end. Advancing to 1504, at least one opening is formed that extends from the cavity through the distal end. Continuing to 1506, an attachment feature may be optionally machined on a surface of the apparatus configured to mate with a corresponding attachment feature of another component. In some embodiments, the apparatus may be a syringe having a nub, and the attachment feature may include a recess configured to mate with a flange or ridge on an interior surface of a cap or sheath (the other component) that also may include a nub. In some embodiments, the apparatus may be a sheath or cap that is configured to couple to a corresponding attachment feature on a syringe. In some embodiments, the sheath and the syringe may include a nub. In some embodiments, the sheath may include a nub, and the syringe may include a neck portion that extends at least partially toward the nub.

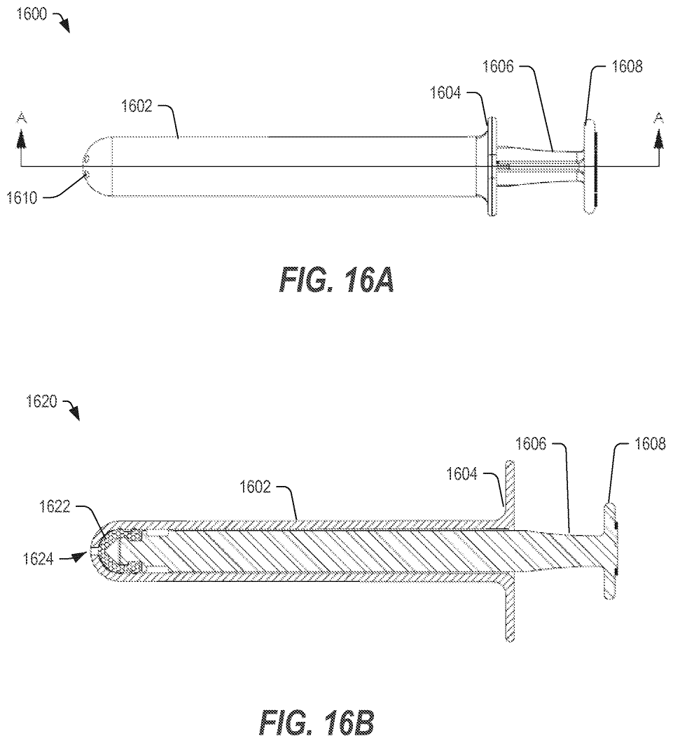

FIG. 16A is a side-view of a syringe 1600 including multiple openings, in accordance with certain embodiments of the present disclosure. The syringe 1600 may include a body portion 1602 having a flange 1604. The body portion 1602 may define a cavity sized to receive a rode portion 1606 of a plunger 1608. The body portion 1602 may further include one or more openings 1610 at a distal end opposite the flange 1604. The one or more openings 1610 may extend from an exterior surface through the distal end of the body portion and into the internal cavity. In some embodiments, the number, the spacing, the shape, and the interior dimensions of the openings 1610 may vary to provide a desired diffusion for fluid disbursed through the openings 1610.

FIG. 16B is a cross-sectional view 1620 of the syringe 1600 of FIG. 16A taken along line A-A, in accordance with certain embodiments of the present disclosure. The syringe 1600 further includes a seal 1622 configured to fit over a tip of the rod portion 1606 of the plunger 1608 to form a fluid seal against the interior surface of the cavity of the body portion 1602 in order to push fluid toward and through the openings 1610. In the cross-sectional view, a central opening 1624 is shown, which may be formed at a substantially central position in the distal end of the body portion 1602 (opposite end from the flange 1604).

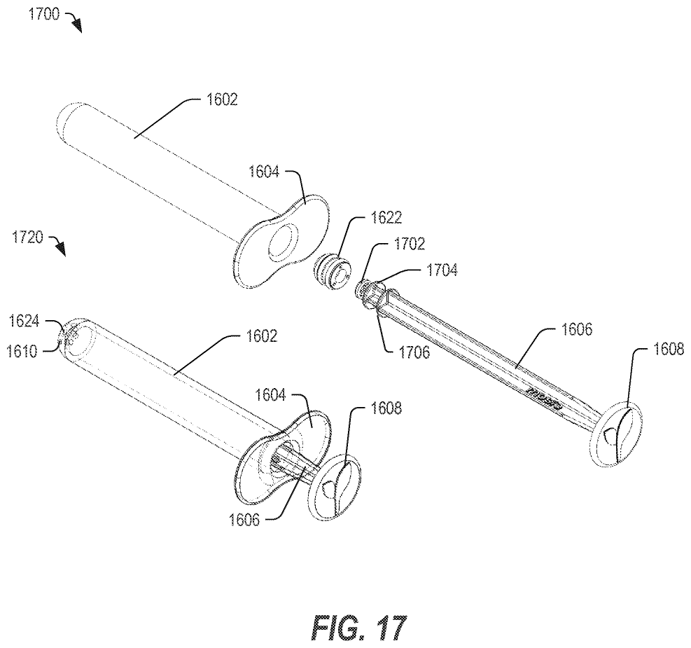

FIG. 17 is an exploded view 1700 and a perspective view 1720 of the syringe 1600 of FIG. 16A, in accordance with certain embodiments of the present disclosure. The exploded view 1700 includes the seal 1622. Further, the exploded view 1700 includes the rod portion 1606 of the plunger 1608. The rod portion 1606 includes an end 1702 sized to fit into an opening of the seal 1622. The rod portion 1606 may further include a flange portion 1702 sized to engage an interior surface of the seal 1622 and may further include a stopper portion 1704 configured to engage a portion of the seal 1622 to prevent over stressing. In some embodiments, the seal 1622 may be installed onto the end 1702 and may be pushed over the flange portion 1702 to engage the stopper portion 1704. The flange portion 1702 may engage an interior portion of the seal 1622 to secure the seal 1622 to the rod portion 1606.

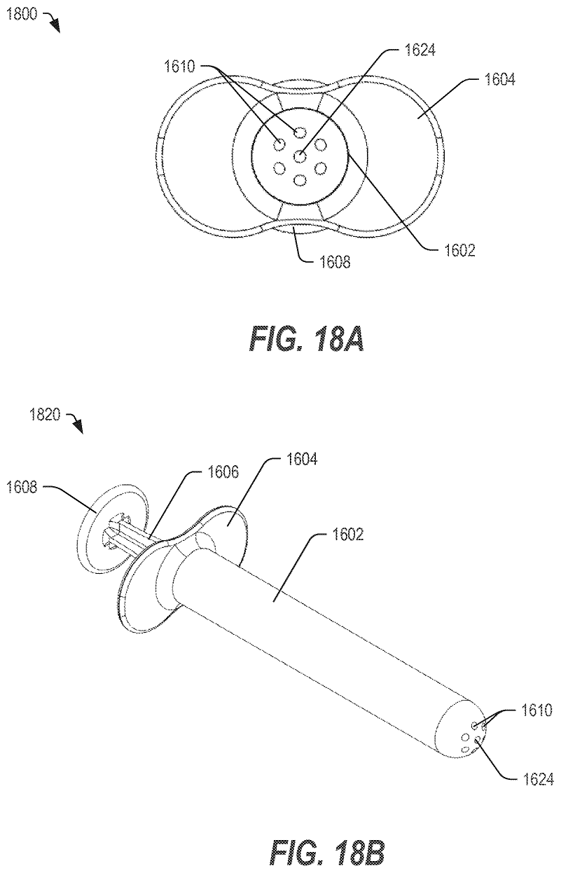

FIG. 18A is a front view 1800 of the syringe 1600 of FIG. 16A, in accordance with certain embodiments of the present disclosure. The front view 1800 depicts the plunger 1608, the flange 1604, and the body portion 1602. The body portion 1602 includes the openings 1610 and the central opening 1624. In the illustrated example, the openings 1610 and 1624 are approximately the same size and shape. However, in other embodiments, the openings 1610 may be a different size and shape as compared to the central opening 1624. In some embodiments, instead of being arranged in a circular formation around the central opening 1624, the openings 1624 may be arranged in a different configuration.

FIG. 18B is a perspective view 1820 of the syringe 1600 of FIG. 16A, in accordance with certain embodiments of the present disclosure. The perspective view 1820 includes the openings 1610 and 1624 at the distal end of the body portion 1602 opposite to the flange 1604.

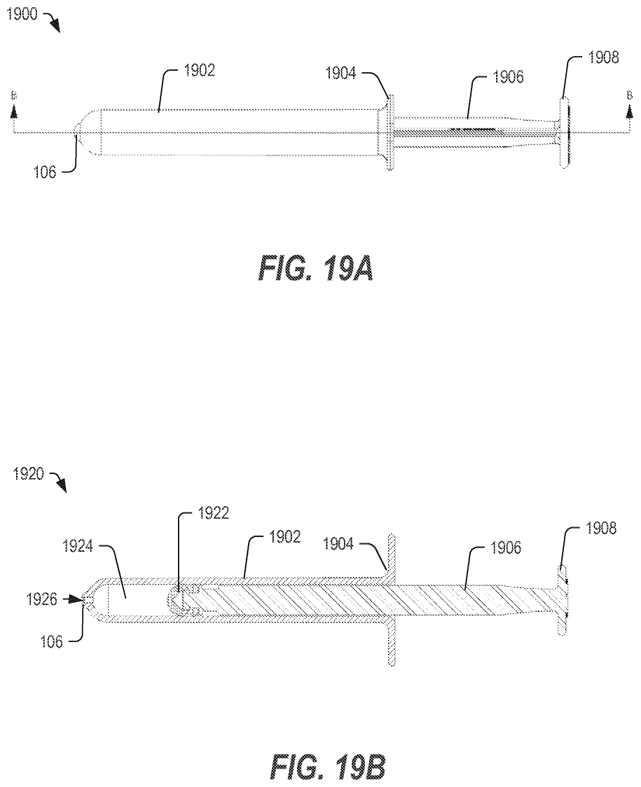

FIG. 19A is a side view of a syringe 1900 including a nub 106, in accordance with certain embodiments of the present disclosure. The syringe 1900 may include a body portion 1902 having a flange 1904. The syringe 1900 may further include a plunger 1908 having a rod portion 1906, which may have an end to which a seal 1922 may be coupled. The syringe 1900 may include an opening that may extend from an exterior surface of the nub 106 into the interior cavity defined by the body portion 1902.

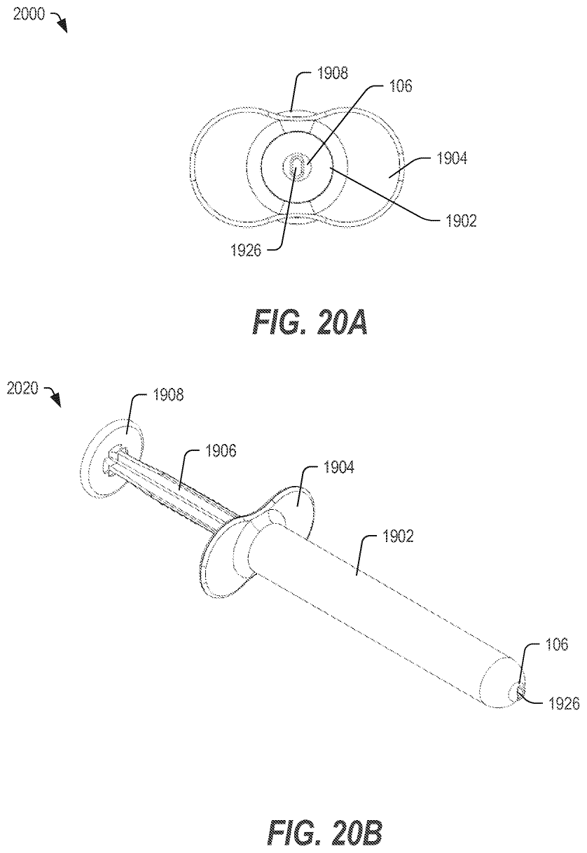

In some embodiments, the opening through the nub 106 may have a substantially irregular shape, which may diffuse or otherwise distribute fluid as it passes through the opening. Examples of such irregular shapes may be described below with respect to FIGS. 20A-22B. As used herein, the term “irregular shape” refers to a shape other than a circle or square shape.

FIG. 19B is cross-sectional view 1920 of the syringe 1900 of FIG. 19A taken along line B-B, in accordance with certain embodiments of the present disclosure. The cross-sectional view 1920 depicts a cavity 1924 within the body portion 1902. The cavity 1924 may hold fluid to be disseminated through the opening 1926, which may extend from the cavity 1924 through the nub 106.

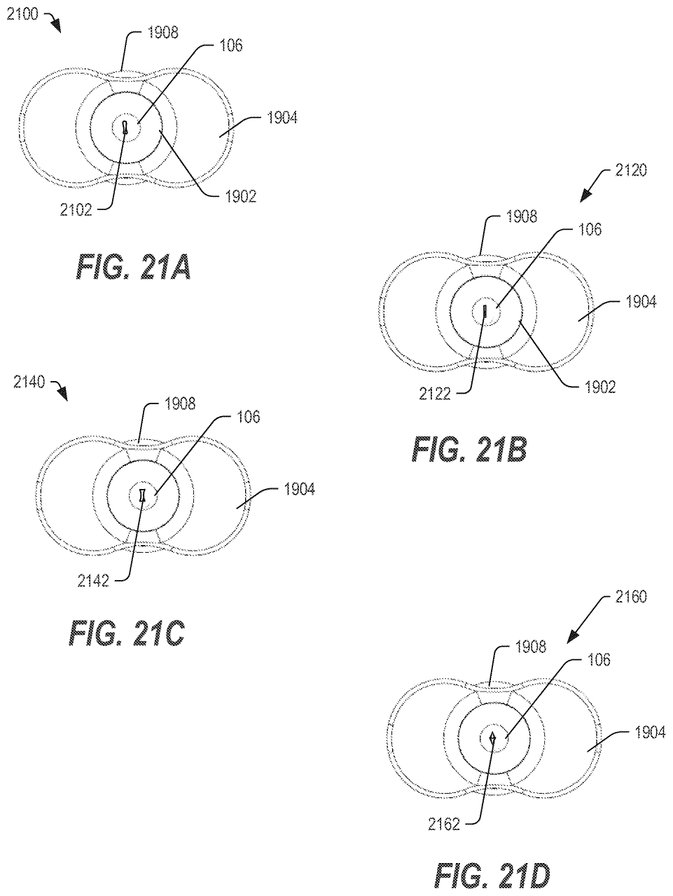

As discussed above, in some embodiments, the body portion 1902 may include a plurality of openings. In some embodiments, the opening 1926 may have a substantially irregular shape. Further, in some embodiments, the opening 1926 may define a fluid passage having an irregular internal shape. Examples of the irregular shaped opening 1926 may be described below with respect to FIGS. 20A-21D.

FIG. 20A is a front view 2000 of the syringe 1900 of FIG. 19A, in accordance with certain embodiments of the present disclosure. The front view 2000 includes the body portion 1902, the flange 1904, the plunger 1908 and the nub 106. In the illustrated embodiment, the opening 1926 may be implemented as a rounded rectangular shape, an elliptical shape, an oval shape, or another non-circular shape. The opening 1926 may extend from the cavity within the body portion 1902 through the nub 106. Other openings may also be provided (either through or around the nub 106).

FIG. 20B is a perspective view 2020 of the syringe 1900 of FIG. 19A, in accordance with certain embodiments of the present disclosure. The perspective view 2020 depicts the opening 1926 having a substantially elliptical, oval or oblong shape, which may provide a diffused fluid dispersion pattern as compared to a high-pressure stream that might be produced by a small circular opening.

FIGS. 21A-21D are front views of the syringe of FIG. 19A, in accordance with certain embodiments of the present disclosure. Referring now to FIG. 21A, a front view of the syringe 1900 of FIG. 19A is shown. The front view includes the body portion 1902, the flange 1904, the plunger 1908 and the nub 106. The nub 106 may include an opening 2102 having an irregular shape, such as a barbell type of shape or another shape.

FIG. 218 depicts a front view 2120 of the syringe 1900 of FIG. 19A including an opening 2122 having an irregular shape that is substantially rectangular-shaped. In some embodiments, the rectangular-shaped opening 2122 may provide a non-uniform dispersal of fluid.

FIG. 21C depicts a front view 2140 of the syringe 1900 of FIG. 19A including an opening 2142 having an irregular shape that is substantially hourglass-shaped. In some embodiments, the hourglass-shaped opening 2142 may provide a non-uniform dispersal of fluid.

FIG. 21D depicts a front view 2160 of the syringe 1900 of FIG. 19A including an opening 2162 having an irregular shape that is substantially diamond-shaped. In some embodiments, the diamond-shaped opening 2162 may provide a non-uniform dispersal of fluid.

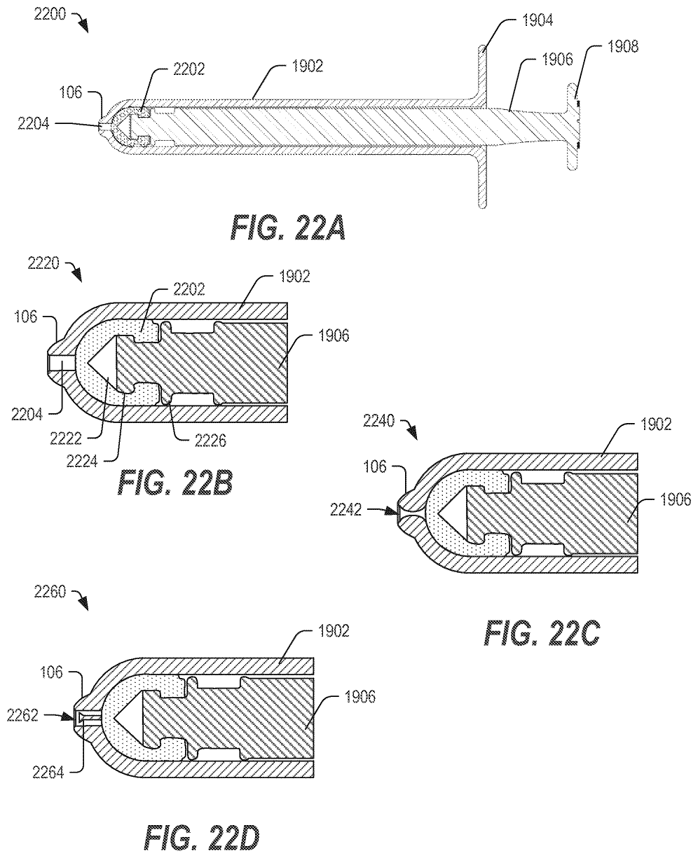

FIG. 22A is a cross-sectional view 2200 of the syringe 1900 of FIG. 19A including an opening 2204. In some embodiments, the opening 2204 may define a cylindrically-shaped fluid passage extending from the cavity within the body portion 1902 through the nub 106. The cross-sectional view 2200 further depicts the rod portion 1906 of the plunger 1908 extending within the cavity of the body portion 1902. Further, the rod portion 1906 may include an end portion 2222, a flange portion 2224, and a stop portion 2226. Further, a seal 2202 may be coupled to the end portion 2222 and may be configured to provide a fluid seal extending a full internal diameter of the cavity to push fluid toward the opening as the plunger 1908 is pushed toward the distal end of the body portion 1902.

FIG. 22B is a cross-sectional view 2220 of a portion of the syringe 1900 including the opening 2204 having a substantially cylindrical fluid passage that extends from an external surface of the nub 106 to a cavity defined by the hub portion of the syringe 1900. In the illustrated example, the rod portion 1906 is fully inserted within the cavity such that the seal 2202 is in contact with the interior surface of the distal end of the body portion 1902.

FIG. 22C is a cross-sectional view 2240 of a portion of the syringe 1900 including an opening 2242 defining a fluid passage having a varying internal diameter. In the illustrated example, the varying internal diameter may form a Venturi tube type of passage having a narrow portion toward the middle of the passage and a wider portion at the opening to provide reduced pressure and increased dispersion.

FIG. 22D is a cross-sectional view 2260 of a portion of the syringe 1900 including an opening 2262 defining a fluid passage. Within the opening 2262, the syringe 1900 may include an obstruction element 2264 configured to diffuse or otherwise disperse fluid that may be pushed through the opening 2262 by the plunger 1908 via the rod portion 1906 and the seal 2202.

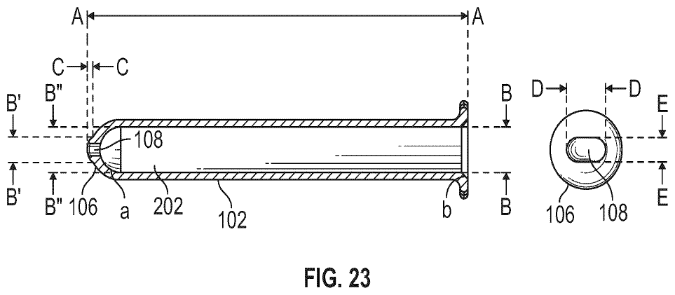

FIG. 23 is a side view cross-sectional view and front view with sectional lines illustrating preferred dimensional measurements of the present syringe assembly. Section A-A is the length of syringe 102. Section B-B is the height or width of the syringe. Section B″-B″ is the is the internal diameter of the syringe barrel. Section B′-B′ is the exterior diameter of the nub 106. Section C-C is the height of the nub 106. Attention is directed in particular to the nub 106. The form of nub 106 is subtle, with smooth curves to facilitate comfortable insertion. Nub 106 is small relative to the length of barrel from which it is form. In an exemplary preferred embodiment, the length of barrel, including nub 106, of section A-A is 3.6 inches (91.4 mm) whereas the length C-C (that is, the height of the nub portion 106 which extends contiguously from the end of barrel), is approximately 0.2 inches (4.9 mm) or in a range of 4 mm-6 mm. The length/height of nub 106, therefore, is approximately 5.4% the length of barrel. An operable preferred embodiment has a nub height in the range of 4% to 6% the length of the barrel. In the same exemplary preferred embodiment, the diameter of nub B′-B′ has a nub exterior diameter in the range of 0.3 to 0.4 inches (7.6 mm-10.1 mm). Opening 108 traverses through nub 106 and has preferred dimensions of 0.09 inches (2.3 mm) by 0.05 inches (1.3 mm).

The relatively small size, dimensions, and smooth curvature of nub 106 and the preferred dimensions of the opening 108 are not purely ornamental, arbitrary or merely a matter of design choice. The embodiment of the preferred dimensions is the result of testing, iteration and refining of the design with the objective of obtaining desired performance characteristics. The performance characteristics include little loss of payload in delivery to the cervix, effective dispersal of the payload, and comfort of use. The small nub is comfortable for the user and provides a very short channel or reservoir in the opening through which the payload is delivered, resulting in very little loss of payload left trapped in the syringe, and a geometry of the opening to provide effective dispersal of the payload into the cervix.

Nevertheless, in some embodiments, the invention contemplates that the shape of the opening, the shape of the fluid passage, or any combination thereof may be selected to provide a desired fluid dispersion at a distal end of the syringe. In some embodiments, the external diameter and length of the syringe may be selected to provide a suitable insertion vessel through which the fluid may be presented. With the use of an insemination syringe, for example, the vagina extends from the vulva to the uterus, and the length of the vagina may vary from about 2.75 inches to about 4.75 inches. Accordingly, the length of the syringe may be selected to be a suitable length for a particular woman. For insemination purposes, the body portion of the syringe (from the distal end to the flange) may be approximately 2.75 to 3 inches in length. Other lengths may also be possible.

In conjunction with FIGS. 1-23, an apparatus is described that may include a sheath or body portion having a distal end having a rounded nub and at least one opening. In some embodiments, the at least one opening may include a non-circular or irregular shaped opening, such as a slit, an hourglass shape, a barbell shape, a diamond shape, another shape, or any combination thereof. The distal end of the apparatus may be rounded to present a blunt or “edgeless” end presenting a substantially smooth transition from an elongate portion to a distal portion of the apparatus. In some embodiments, the apparatus may be used for home-based artificial insemination, such as by insertion of the apparatus into the vagina of a woman until the nub contacts the woman's cervix. The plunger may then be depressed to expel seminal fluid onto the cervix through the opening to provide artificial insemination.

In some embodiments, the apparatus may include a syringe having a substantially tubular shape and having a substantially rounded distal end. The syringe may further include a rounded nub extending from the distal end proximate to a longitudinal axis of the syringe. In some embodiments, the rounded nub may include the opening to allow fluid to pass therethrough. In some embodiments, the distal end may include a plurality of openings to allow fluid passage. In some embodiments, the opening or openings in the rounded nub, the other openings at the distal end, or any combination thereof may have non-circular or irregular shapes to diffuse the fluid as it passes through the one or more openings.

In some embodiments, the apparatus may include a syringe and a sheath having a cavity sized to receive the syringe. The sheath may include one or more openings on a distal end that are configured to align with (or mate with) a fluid opening of the syringe. In some embodiments, the sheath may include an attachment feature configured to mate with a corresponding feature on the syringe. In an example, the attachment feature may include threads configured to mate with corresponding threads of the syringe to secure the syringe within the sheath. In another example, the attachment mechanism may include a raised edge on one of an interior surface of the sheath and an exterior surface of the syringe that is configured to mate with a corresponding recess on the other of the exterior surface of the syringe and the interior surface of the sheath. Further, in some embodiments, at least a portion of the sheath may be formed from a first material, and at least a portion of the syringe may be formed from a second material. In some embodiments, the first material may be more flexible or malleable than the second material.

In some embodiments, a cap or partial sheath may be configured to mate with a syringe to provide a rounded end having a nub and including one or more openings for fluid passage. In some embodiments, the nub may be rounded and may include an opening configured to align to an opening of the syringe to allow fluid from the syringe to pass there-through. The opening of the cap or partial sheath may be irregular in shape (e.g., oval, elliptical, hourglass shaped, barbell shaped, rectangular shaped, or some other shape). In some embodiments, the cap or partial sheath may include an attachment feature configured to mate with a corresponding feature of the syringe to secure the cap or partial sheath to the syringe. In some embodiments, the syringe may be formed from a first material, and the cap or partial sheath may be formed from a second material. The first material may be more rigid than the second material.

Referring to FIGS. 24-33B, the collection cup 10 includes a container 14 and a lid 18, and the collection cup 10 may be used to collect, transport, and store a fluid sample (e.g., semen, saliva, urine, etc.). For example, the collection cup 10 may be part of an at-home insemination kit including a syringe. The collection cup 10 can be used to collect and transport semen, and the syringe can be used to collect the semen from the collection cup 10. The collection cup 10 may have an interior shape to facilitate easy collection of fluid and easy withdrawal from the container 14 via a syringe.

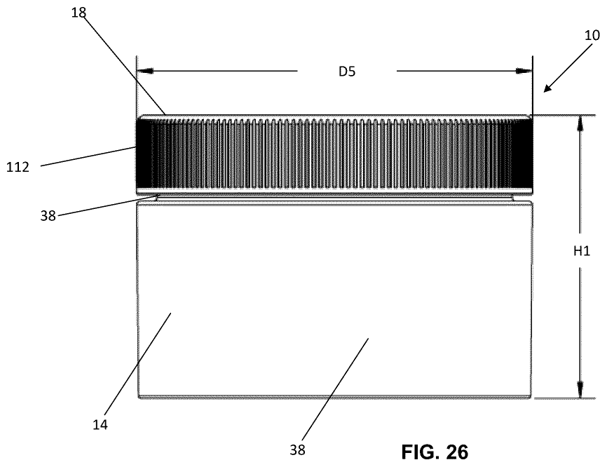

With reference to FIG. 26, the collection cup 10 has a total height H1, which includes the container 14 and the lid 18, of approximately 1.5-1.6 inches. The total height H1 may be between approximately 1.2 inches and approximately 5 inches. In some constructions, the total height Hl may be between approximately 1.5 inches and approximately 2 inches, or above 5 inches.

The container 14 may be formed from a thermoplastic polymer such as polystyrene or Acrylonitrile Butadiene Styrene (ABS). The polystyrene may be transparent such that it is easy to identify contents of the container 14. The container 14 has a container height H2 of approximately 1.4-1.5 inches. In some constructions, the container height H2 may be between 1.1 inches and 2 inches, or approximately 5 inches or higher. The container height H2 is less than the total height H1. In some constructions, the container 14 may be formed from an opaque thermoplastic polymer, such as ABS.

The container 14 includes an outer wall 22 and an inner wall 26 that extends from the outer wall 22 and that defines a storage space for fluid. The outer wall 22 has a top or first region 30, a bottom or second region 34 opposite the top region 30, and a step or transition 38 that interconnects the top region 30 and the bottom region 34. The bottom region 34 is defined by a cylindrical shape and has a lower edge that defines a base 39 (e.g., for supporting the container 14 on a surface such as a table or counter). The illustrated outer wall 22 and the inner wall 26 are monolithic (e.g., formed together in the same process as one piece, although the outer wall 22 and the inner wall 26 may be formed in other ways (e.g., the inner wall 26 may be inserted into the outer wall 22 after the outer wall is formed).

In the top region 30, the inner surface 26 may be flush with the outer surface 22 or have a thickness that is thinner than a thickness of the top region 30 adjacent the transition 38. The outer wall 22 may be cylindrical or substantially cylindrical on the outside and may have an inner taper 96 extending inward toward the center of the container 14 such that a gradual vertical taper TR1 exists (e.g., between approximately 0.2-0.6 degrees).

The top region 30 defines an opening 40 and has a top height H3 between approximately 0.2 inches and 0.8 inches. The ratio between the total height H1 of the container 14 and the top height H3 of the top region 30 is between approximately 2:1 and 4:1. The top region 30 has an outer diameter D1 and the opening has a diameter D2 smaller than the diameter D1 such that the top region 30 has a wall thickness T1 between approximately 0.1 inches and approximately 0.2 inches, or below 0.1 inches (e.g., 0.06-0.1 inches). The diameter D2 may be between approximately 1.7 inches and 2 inches. In some constructions, the diameter D2 between 1.9 inches and 2 inches, or below 1.7 inches or above 2 inches.

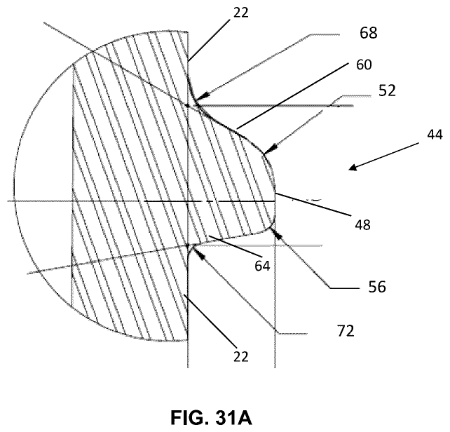

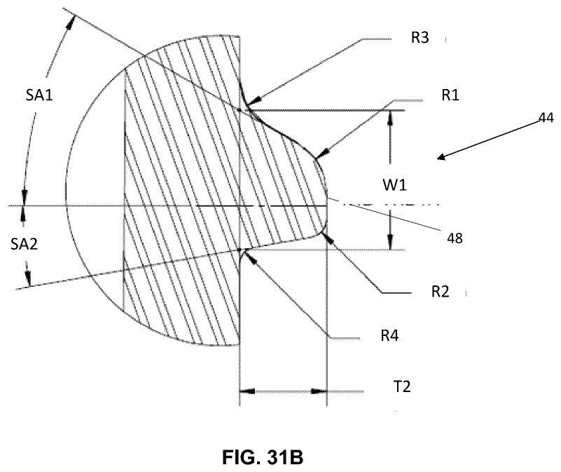

The top region 30 has a threaded region 44 that is engageable by an internal thread on the lid 18. The threaded region 44 may be disposed around the entirety of the top region 30 or in segments around the top region 30. With reference to FIGS. 31A-31B, the threaded region 44 includes a ridge 48 that protrudes from the outer wall 22. The ridge 48 may have a ridge thickness T2 between approximately 0.04 inches to 0.05 inches and a width W1 between 0.07 inches and 0.08 inches. The ridge 48 may have a first rounded edge 52 (e.g., a top rounded edge) and a second rounded edge 56 (e.g., a bottom rounded edge). In the illustrated construction, the first rounded edge 52 has a larger radius than the second rounded edge 56. More specifically, the first rounded edge 52 has a radius R1 that is two to three times larger than the radius R2 of the second rounded edge 56. The first rounded edge 52 may have a radius R1 between 0.025 in and 0.035 inches. The second rounded edge 56 may have a radius R2 between 0.05 in and 0.018 inches. It will be appreciated that in other constructions the second rounded edge 56 may have a larger radius than the radius of the first rounded edge 52.

The ridge 48 protrudes from the outer wall 22 along a first slope 60 and extends toward the outer wall 22 along a second slope 64. The first slope 60 may extend from a leading edge 68 to the first rounded edge 52, and the second slope 64 may extend from the second rounded edge 56 to a trailing edge 72. The second slope 64 may be steeper than the first slope 60. More specifically, the second slope 64 may be two to three times steeper than the first slope 60. The first slope 60 may have a slope angle SA1 between 25 and 35 degrees. The second slope 64 may have a slope angle SA2 between 5 and 18 degrees. It will be appreciated that in other constructions the first slope 60 may be steeper than the second slope 64. The leading edge 68 and the trailing edge 72 may have rounded edges such that there is a smooth transition between the outer wall 22 and the ridge 48. The radius R3 of the leading edge 68 may correspond with the radius R1 of the first rounded edge 52, and the radius R4 of the trailing edge 72 may correspond with the radius R2 of the second rounded edge 56.

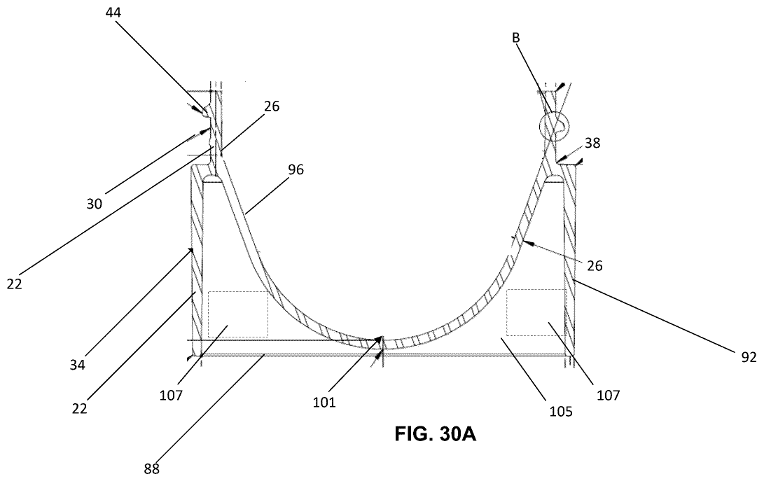

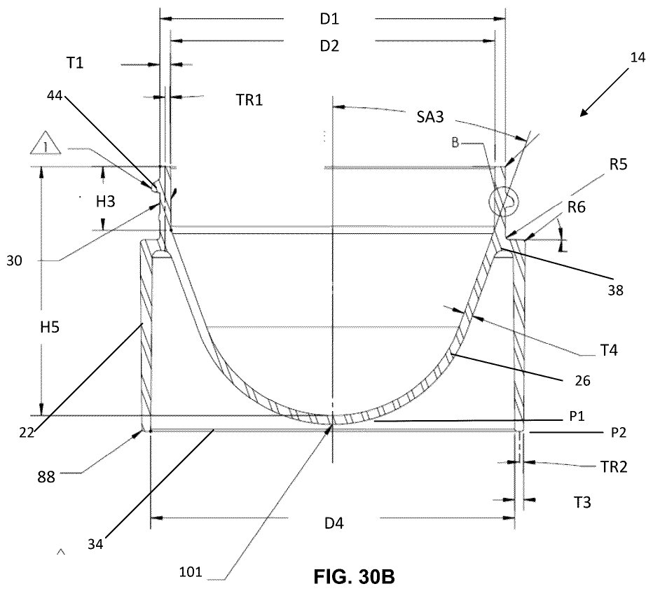

At the bottom region, the outer wall 22 has a diameter D3 that is larger than the top diameter D1 of the top region 30. The diameter D3 may between approximately 1.8 inches and approximately 2.3 inches. In some constructions, the diameter D3 may be between approximately 2 inches and approximately 2.2 inches. With reference to FIG. 30A, the bottom region 34 has a height H4 that extends from the transition 38 to the base 39. The height H4 is between approximately 0.5 inches and approximately 1.5 inches. The outer wall 22 may have an outer wall thickness T3 between approximately 0.04 in and approximately 0.06 inches. The outer wall thickness T3 of the outer wall 22 in the bottom region 34 may be the same as, thicker, or thinner than the wall thickness T1 of the top region 30. In some constructions, the outer wall thickness T3 may be greater than 0.06 inches.

The illustrated outer wall 22 defines a cylindrical wall 92, although the wall 22 may have a taper TR2 (e.g., inward toward the center of the container 14). For example, the taper TR2 may be between approximately 0.2-0.6 degrees. The taper TR2 means that the base 88 to have a smaller base diameter D4 than the neck diameter D3. The base diameter D4 may be larger than the top diameter D1. For example, the base diameter D4 may be between approximately 1.8 inches and approximately 2.3 inches. In some constructions, the base 39 may have a base diameter D4 between approximately 2 inches and approximately 2.2 inches.

The inner wall 26 may be flush or merge with the outer wall 22 adjacent or in the top region 30 (e.g., at or adjacent the transition 38). The inner wall 26 is defined by a tapered wall section 96 (e.g., defining a frusto-conical shape) that terminates in a curved lower section 78 defining a receptacle or accumulation area 101. The tapered wall section 96 tapers inward toward the center of the container 14 (e.g., at an angle SA3 of approximately 5-30 degrees (e.g., 20 degrees) from a vertical axis or plane bifurcating the container 14). The tapered wall section 96 may have a curvature toward the lower section. The inner wall 26 may have an inner wall thickness T4 that corresponds to the outer wall thickness T3 of the outer wall 22. The inner wall thickness T4 may be between approximately 0.4 inches and 0.6 inches.

The illustrated accumulation area 101 is semi-spherical, although the accumulation area may have other curvatures (e.g., an inverse dome or elliptical shape). The curvature of the accumulation area 101 allows fluid to be easily retrieved by a syringe. A horizontal plane P1 extending through the bottom-most part of the accumulation area 101 (on the outer side of the inner wall 26, through a tangent with the inner wall 26) may be spaced from a plane P2 extending through the edge or base 39 (each plane P1, P2 is shown horizontal as viewed in FIG. 30B). The inner wall 26 is separated from the outer wall 22 by a space 105 due to the taper and curvature. In some embodiments, an insulative material 107 (shown schematically in FIG. 30A) may be disposed in the space 105. The insulative material 107 may be optional or may be connected to one or both of the outer wall 22 and the inner wall 26. In some constructions, the outer wall 22 may have a larger outer wall thickness T3 and the base 88 may have a larger thickness such that there is no space between the inner wall 26 and the outer wall 22.

The lid 18 encloses or covers the container 14 and is removably coupled to the threaded region 44 of the container 14. More specifically, the lid 18 includes an internal threaded region (not shown) that engages with the threaded region 44. The lid 18 may form an airtight or fluid-tight seal with the container 14 to prevent fluid from entering or exiting the container 14. The lid 18 may be made from a thermoplastic material such as polypropylene. The lid 18 may have a lid height H6 that is similar to the top height H3 of the container 14. More specifically, the lid height H6 may be slightly larger (e.g., less than 0.05 in) than the top height H3 of the top region 30. The lid diameter D5 of the lid 18 may be substantially similar to the diameter of the widest portion of the container 14. More specifically, the lid diameter D5 of the lid 18 may be substantially similar to the neck diameter D3. It will be appreciated that the lid diameter D5 may be larger than the widest portion of the container 14.

The lid 18 may include a top surface 109 and a ribbed side surface 113. The top surface 109 may be smooth or the top surface 109 may include a stippled texture. It will be appreciated that the ribbed side surface 113 may extend the entire circumference of the lid 18 or the ribbed side surface 113 may only partially extend along a portion of the circumference of the lid 18. The ribbed side surface 113 provides a grip so that the lid 18 can be easily removed from or tightened onto the container 14.

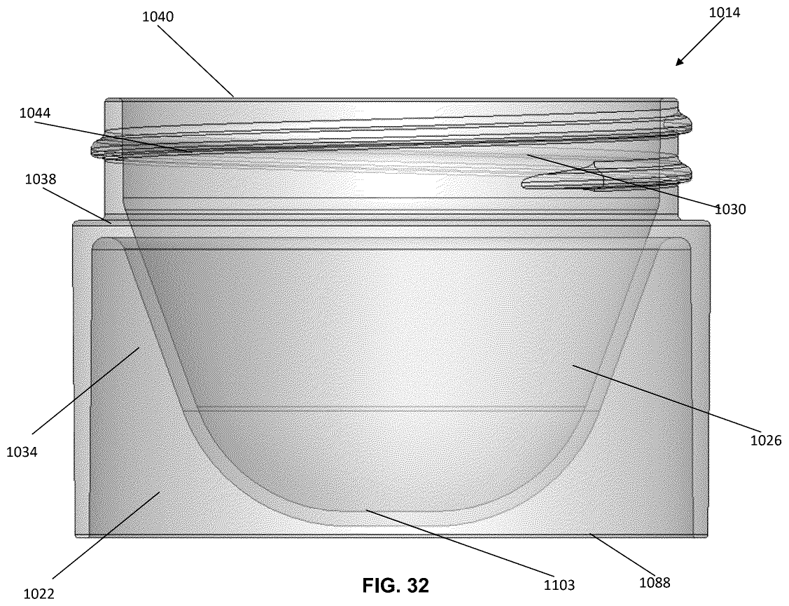

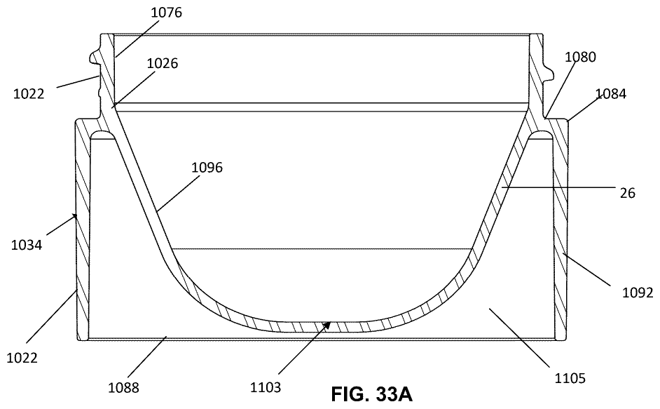

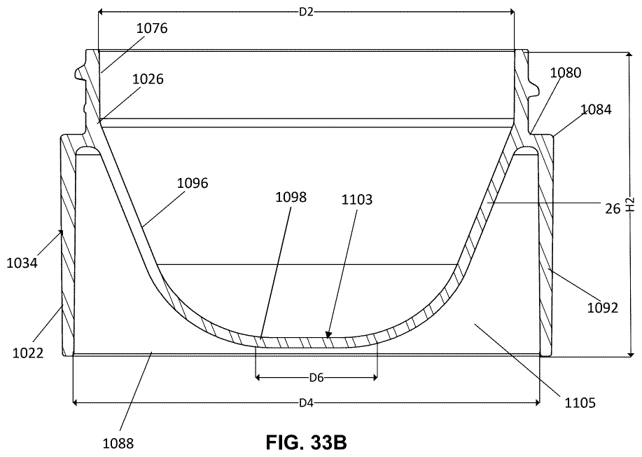

FIGS. 32-33B illustrate another construction of a container 1014 that may be used with the lid 18 to form the collection cup 10. Many features of the container 1014 are similar to or the same as those features described above with regard to the container 14. Features that are similar to or the same as those described above will be labeled with a reference number that is a value of a thousand higher than the corresponding feature described above.

The container 1014 may be formed from a thermoplastic polymer such as polystyrene. The polystyrene may be transparent such that it is easy to identify if there is a fluid in the container 1014. The container 1014 may have the same container height H2 (FIG. 33B) as the container 14, or a different height. The container 1014 includes an outer wall 1022 and an inner wall 1026 disposed inside the outer wall 1022. A top region 1076 extends from the outer wall 1022 and the inner wall 1026. The structures and features of the outer wall 1022 are the same as the outer wall 22. The container 1014 also includes a top region 1030, a bottom region 1034, and a transition 1038 that connects the top region 1030 to the bottom region 1034. The bottom region 1034 extends from the transition 1038 to a base 1039. The container 1014 may be integrally formed such that the outer and inner walls 1022, 1026 are one solid piece (e.g., formed from the same material at the same time, or from different material at the same time).

The top region 1030 defines an opening 1040 that has an opening diameter D2 (FIG. 33B) between approximately 1.6 inches and approximately 2.2 inches (e.g., between approximately 1.7 inches and approximately 2 inches, such as between 1.8 inches and 1.9 inches). In the top region 1030, the outer wall 1022 may include a threaded region 1044 that engages an internal thread on the lid 18.

With reference to FIGS. 33A-33B, the inner wall 1026 is configured to hold the fluid and may be flush or merge with the outer wall 1022 adjacent or in the top region 1030 (e.g., at or adjacent the transition 1038). The inner wall 1026 is defined by a tapered wall section 1096 (e.g., defining a frusto-conical shape) that merges with a lower section 1098, which defines a receptacle or accumulation area 1103. The tapered wall section 1096 tapers inward toward the center of the container 14 (e.g., at an angle SA3 of approximately 5-30 degrees (e.g., 20 degrees) from a vertical axis or plane bifurcating the container 14). The tapered wall section 1096 may have a curvature toward the lower section 1078. The inner wall 26 may have an inner wall thickness T4 that corresponds to the outer wall thickness T3 of the outer wall 1022. The inner wall thickness T4 may be between approximately 0.4 inches and 0.6 inches.

The illustrated lower section 1098 with the accumulation area 1103 is defined by a curved-flat profile such that, as the inner wall 1026 extends downward, the inner wall 1026 transitions from an angled-flat profile (defined by the tapered wall section 1096) to a curved profile 1080 (e.g., semi-spherical), and then to a flattened profile 1084 (e.g., a planar section or a shallow curvature, or a combination thereof), although the accumulation area may have combinations of flat and/or curvatures. In one example, the curved-flat profile may have a parabolic or substantially parabolic shape. The curvature of the accumulation area 101 allows fluid to be easily retrieved by a syringe and, as shown, the flattened profile 1084 is larger than an end of the syringe taking up fluid in the accumulation area 1103 (e.g., to facilitate easier take-up of fluid). The flattened profile 1084 has a diameter D6 that is smaller than the base diameter D4 (e.g., the diameter D6 may be approximately or less than 50% of the diameter D4). In some constructions, the diameter D6 may be less than 25% of the diameter D4. In some constructions, the diameter D6 may be less than 15% of the diameter D4 (e.g., approximately or less than 10% of the diameter D4. The diameter D6 may be between approximately 0.3 inches and 1.5 inches.

The lower sections 78, 1078 can have different profiles to facilitate accumulation of fluid within the container 14, 1014. The examples illustrated in the Drawings and described herein are non-limiting and it should be appreciated that variations among flatness or planar tapering associated with the inner wall 1026, as well as curvatures of some or all of the inner wall, are possible and considered herein. The remaining features of the container 1014 are the same as described with regard to the container 14.

It will be appreciated that the container and the lid may have measurements (e.g., height, wall thickness, diameter) outside of the ranges that have been described herein.

Although the present invention has been described with reference to preferred embodiments, workers skilled in the art will recognize that changes may be made in form and detail without departing from the scope of the invention. For example, the syringe, the plunger, and the cup may define a system by which the cup accumulates a fluid, and the syringe and plunger cooperate to withdraw fluid from the cup. As will be appreciated. the cup has an inner profile that is shaped to permit full withdrawal of the collected fluid.

Various features and advantages of the invention are set forth in the following claims.

1. A sample collection cup comprising:
a top region defining an opening having a top diameter;
a bottom region opposite the top region and defining a base;
an outer wall extending from the top region to the bottom region; and
an inner wall coupled to the outer wall and extending inward away from the outer wall and downward toward the base, the inner wall defining an accumulation area and having a profile configured to direct fluid into the accumulation area.

2. The sample collection cup of claim 1, further comprising insulation disposed between the outer wall and the inner wall.

3. The sample collection cup of claim 1, wherein the outer wall is cylindrical and the inner wall is non-cylindrical.

4. The sample collection cup of claim 3, wherein the inner wall has a parabolic shape.

5. The sample collection cup of claim 3, wherein the inner wall has a semi-spherical shape.

6. The sample collection cup of claim 1, wherein the accumulation area is at least partially defined by a flat section of the inner wall.

7. A sample collection cup comprising:
a top region defining an opening and a top diameter;
a bottom region opposite the top region;
a transition connecting the top region and the bottom region; and
at least one wall including an outer surface extending from the top region to the bottom region and an inner surface extending from the transition toward the bottom region, wherein the inner surface has a lowermost flat section.

8. The sample collection cup of claim 7, wherein the transition defines a neck diameter that is different from the top diameter.

9. The sample collection cup of claim 7, wherein the at least one wall includes a first wall defining the outer surface and a second wall different from the first wall and defining the inner surface, and wherein the first wall defines a base configured to support the sample collection cup on a surface.

10. The sample collection cup of claim 9, wherein a first plane extends through an end of the first wall at the base and a second plane extends tangentially through an outer surface of the second wall at the bottom region, and wherein the second plane is spaced from the first plane.

11. The sample collection cup of claim 9, wherein the first wall is substantially cylindrical and wraps around the second wall, and wherein the first wall is open-ended at the bottom region.

12. The sample collection cup of claim 9, wherein the first wall and the second wall join at a lower end of the top region.

13. The sample collection cup of claim 7, wherein the lowermost flat section has a diameter that is less than 25% of a diameter of the outer surface at the bottom region.

14. A sample collection system comprising:
a syringe including a body elongated along a longitudinal axis, the body having an open end and an opening defined on a distal end of the body opposite the open end;
a plunger configured to extend into the open end and along the longitudinal axis toward the opening; and
a cup including a first wall defining an outer profile and a second wall inward of the first wall and defining an inner profile different from the outer profile, the inner profile shaped to accumulate fluid in the cup,
wherein the plunger is configured to draw fluid accumulated in the cup into the syringe.

15. The sample collection system of claim 14, wherein the body tapers narrower from the open end toward the distal end.

16. The sample collection system of claim 14, wherein the inner profile has a curved-flat shape with a lowermost flat section.

17. The sample collection system of claim 16, wherein the lowermost flat section has a diameter that is less than 25% of a diameter of the outer profile.

18. The sample collection system of claim 16, wherein a first plane extends through an end of the first wall at a bottom region of the cup, and a second plane extends tangentially through an outer surface of the second wall at the bottom region, and wherein the second plane is spaced from the first plane.

19. The sample collection system of claim 14, wherein the first wall is substantially cylindrical and wraps around the second wall, and wherein the first wall is open-ended at a bottom region of the cup.

20. The sample collection system of claim 14, wherein the second wall has a parabolic shape.
